(19) United States
(12) Patent Application Publication
(10) Pub. No.: US 2022/0022929 A1
(43) Pub. Date: Jan. 27, 2022

(54) Medical Fastener Device

(71) Applicants: Nicholas Cordaro, Encinitas, CA (US); Andres Talamantes, Vista, CA (US)

(72) Inventors: Nicholas Cordaro, Encinitas, CA (US); Andres Talamantes, Vista, CA (US)

(21) Appl. No.: 17/381,640

(22) Filed: Jul. 21, 2021

Related U.S. Application Data
(60) Provisional application No. 63/054,945, filed on Jul. 22, 2020.

(51) Int. Cl. A61B 17/86; B33Y 80/00

(52) U.S. Cl. CPC A61B 17/869; A61B 17/864; A61B 2017/00526; A61B 17/866; B33Y 80/00; A61B 17/861

(57) ABSTRACT

An improved bone screw device having an internal lattice structure for bone integration and to promote bony ingrowth. The improved bone screw device has a cylindrical body with a first end and a second end and a cutting flute. The head of the bone screw is configured to engage with a driver to advance the screw into a patient's bone, and a distal tip is positioned at the second end. The cylindrical body further includes an internal lattice structure formed at a base of the cylindrical body, near the distal tip. Further, an exterior thread is formed on the cylindrical body and helically surrounds the internal lattice structure. In use, bone is channeled into the internal lattice structure, which helps to fixate the distal tip of the bone screw into the patient's pedicle, bone or joint.

### CROSS-REFERENCE TO RELATED APPLICATION

The present application claims priority to and the benefit of U.S. Provisional Application No. 63/054,945 filed on Jul. 22, 2020, which is incorporated herein by reference in its entirety.

### FIELD OF THE INVENTION

The present invention relates generally to medical fasteners such as a surgical bone screw and other rigid type fasteners with an internal lattice structure to facilitate healing. More specifically, the internal lattice structure of the bone screw provides for bone integration while in vivo, thereby helping to fixate or otherwise secure the tip of the bone screw into the patient's pedicle. Accordingly, the present specification makes specific reference thereto. However, it is to be appreciated that aspects of the present invention are also equally amenable to other like applications, devices and methods of manufacture.

### BACKGROUND OF THE INVENTION

By way of background, various types of fasteners, such as rods, pins, screws, clips and the like are used to either attach implants and other devices to bone, or to secure damaged bones such as those which have been fractured or otherwise splintered. For example, in the spinal field, surgical bone screws or pins are commonly used to attach plates, rods and other types of implants and devices to one or more of a patient's vertebrae to hold the vertebrae in a desired position or to correct a previous deformity. Specifically, bone screws or pins are often used in orthopedic surgery to secure bone sections to each other or to artificial joints, plates or other structural members or medical components to be retained in place. The bone screws or pins usually have a head with an engagement feature for receiving a driver tool and a threaded shank portion, which is then threaded into the patient's bone.

When these bone screws or fasteners are disposed in or placed adjacent to bony surfaces, it is desirable that sufficient friction be present between the bone screw or fastener and the surface of the bone to secure the bone screw or fastener in place within the bone, and to insure that the bone surface(s) are available for bony fixation, integration and ingrowth over time. Accordingly, surgical implant devices such as bone screws often incorporate mechanically-manufactured friction or other engaging surfaces that are formed by conventional practices such as additive or subtractive manufacturing processes, or utilize friction coatings or chemical or other bonding agents for such purposes. However, these mechanically-manufactured friction surfaces, which typically consist of teeth, grooves, striations, or the like, are often not adequate and do little to promote bony fixation and ingrowth. Similarly, these friction coatings or bonding agents may delaminate and fail over time and are therefore also undesirable.

Consequently, there is a long felt need in the art for an improved bone screw or other medical fastener that incorporates essentially integrally formed friction engaging surfaces that are strong and durable, and which provide adequate surface engagement areas for bony fixation, integration and ingrowth, while still being economical to manufacture and safe and easy to use. There is also a long felt need in the art for an improved bone screw that permits bone growth within the internal lattice structure of the improved bone screw. Finally, there is also a long felt need in the art for an improved bone screw or medical fastener that is relatively inexpensive to manufacture and that is both safe and easy to use.

The present invention discloses an improved bone screw or medical fastener device that has an internal lattice structure in the distal tip area so that bone material is deliberately channeled into the internal lattice structure of the screw or medical fastener, thereby helping anchor the tip of the improved bone screw into a patient's pedicle or other bone or joint area. The improved bone screw or medical fastener further incorporates essentially-integral frictional engagement surfaces that are strong and durable, and that provide adequate surface area for bony fixation and ingrowth.

While this specification makes specific reference to the bone screw device of the present invention as a way in which to prevent screw back-out and provide for bone integration, it will be appreciated by those of ordinary skill in the art that aspects of the present invention are also equally amenable to other like applications and other such medical devices and/or issues.

### SUMMARY OF THE INVENTION

The following presents a simplified summary in order to provide a basic understanding of some aspects of the disclosed innovation. This summary is not an extensive overview, and it is not intended to identify key or critical elements or to delineate the scope thereof. Its sole purpose is to present some concepts in a simplified form as a prelude to the more detailed description that is presented later.

The subject matter disclosed and claimed herein, in one aspect thereof, comprises a bone screw or other medical fastening device having an internal lattice structure for bone integration and to help promote bony ingrowth, and thereby more securely anchoring the medical fastener into the bone. More specifically, the improved bone screw or medical fastener device comprises a cylindrical body having a first end, a second end and an exterior cylindrical surface portion. The cylindrical body further comprises an exterior thread disposed on and covering at least a portion of the exterior cylindrical surface portion of the cylindrical body. A head is positioned at the first end and is configured to engage with a driver or other insertion tool to advance the bone screw or medical fastener into the patient's bone (or withdraw the improved bone screw or fastener from the patient's bone). The bone screw or medical fastener further includes a distal tip positioned at the second end, opposite the head. Further, a lattice structure is formed at a base of the cylindrical body, near the distal tip at the second end. The exterior thread helically surrounds the internal lattice structure which is disposed on the surface between the individual threads. The cylindrical body, the head, the tip and the internal lattice structure are preferably formed as a single integral component, unit or device.

In an alternative embodiment, the present invention may comprise a bone screw or medical fastener device having an internal lattice structure on the cylindrical body, not just near the distal tip at the second end. The internal lattice structure may be applied in different zones of the body of the screw or fastener, or may be applied along the majority of the length of the screw or fastener. Having the internal lattice structure positioned throughout the cylindrical body, as opposed to just at the distal tip, promotes bone growth and helps channel bone material into the internal lattice structure, thereby helping to secure the bone screw.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of the improved bone screw or medical fastener device of the present invention in accordance with the disclosed specification, wherein the bone screw or medical fastener device includes an internal lattice structure;

FIG. 2 shows a perspective close-up view of one potential embodiment of the distal tip of the improved bone screw or medical fastener device of the present invention comprising an internal lattice structure in accordance with the disclosed structure; and

FIG. 3 provides a view of the bone screw or medical fastener showing the lattice structure provided on only a portion of the body of the screw or fastener and the threads covering only a portion of the body of the screw or fastener.

### DETAILED DESCRIPTION OF THE INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

Generally stated, and in one embodiment thereof, the present invention discloses an improved bone screw or medical fastener device having an internal lattice structure for bone integration and to promote healing. More specifically, the improved bone screw device includes a cylindrical body with the internal lattice structure formed at a base of the cylindrical body, near the distal or end tip. Further, a thread or other engagement feature is formed on the external surface of the cylindrical body, and helically surrounds the internal lattice structure. The engagement surface includes one or more bone-cutting flutes. In use, bone material is channeled into the internal lattice structure, which helps to secure the distal tip of the improved bone screw into the patient's pedicle, bone, joint or other area selected for securement.

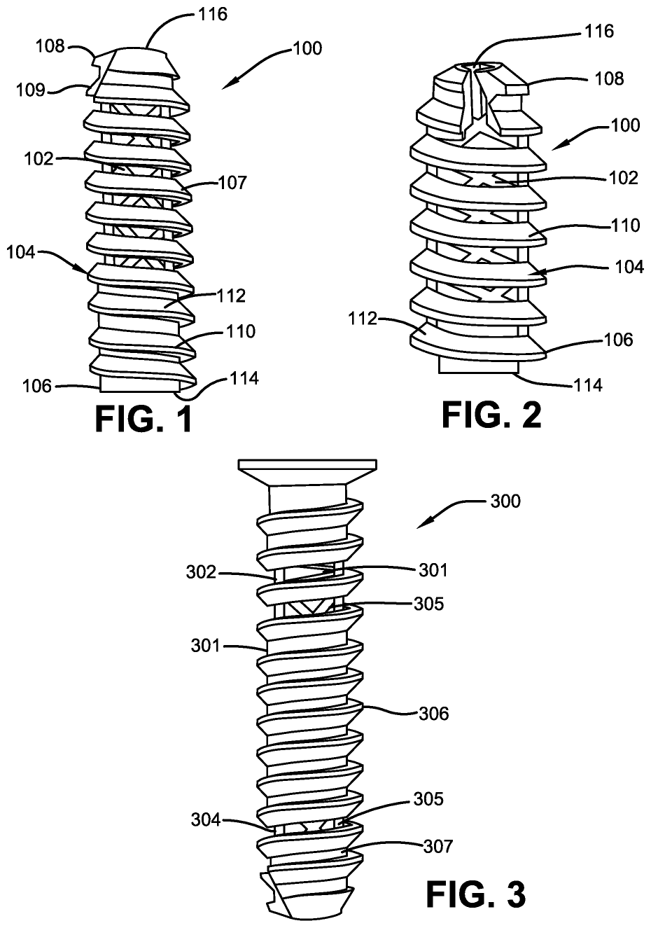

Referring initially to the drawings, FIG. 1 illustrates a perspective view of an improved bone screw or mechanical fastener device 100 comprising an internal lattice structure 102. The bone screw or medical fastener device 100 is typically manufactured using additive manufacturing (AM) techniques and grown as a single integral device. Additionally, the improved bone screw or medical fastener device 100 and its various components can be any suitable size, shape, and/or configuration as is known in the art without affecting the overall concept of the invention. One of ordinary skill in the art will appreciate that the shape and size of the improved bone screw device or medical fastener 100, as shown in the FIGS. 1-2, is for illustrative purposes only, and many other shapes, sizes and/or configurations of the improved bone screw device 100 are well within the scope of the present disclosure. Although the dimensions of the improved bone screw device 100 (i.e., length, width, and height) are important design parameters for good performance, the improved bone screw device or medical fastener 100 may be of any shape, size and/or configuration that ensures optimal performance during implantation and subsequent use.

As shown in FIG. 1, the bone screw device or medical fastener 100 further comprises a generally cylindrical body 104 having a first end 106 and a second end 108. The cylindrical body 104 further includes an exterior thread 110 on at least part of an exterior surface portion 112 of the cylindrical body 104, one or more cutting flutes 109, a head 114 positioned at the first end 106 and configured to engage with a driver or other insertion tool (not shown) to advance or withdraw the bone screw device or medical fastener 100 into or out of the bone and a distal tip 116 positioned at the second end 108 opposite the first end. Further, in a preferred embodiment of the present invention, an internal lattice structure 102 is formed at a base of the cylindrical body 104, near the distal tip 116 at the second end 108. The cylindrical body 104, the cutting flutes 109, the head 114, the distal tip 116 and the internal lattice structure 102 are preferably formed as a single integral device and grown together. For example, the cylindrical body 104, cutting flutes 109, head 114, distal tip 116 and the internal lattice structure 102 can be grown or produced via additive manufacturing techniques. The size of the threads as well as the internal lattice area can vary in size, height or width to create different screw or fastener configurations depending on the surgical or medical procedure the fastener is to be used in connection with. The height of the threaded portion will generally be greater than the height of the lattice work which sits between the threads so that the threads may still cut into the bone as it is being inserted and the lattice work will not interfere with the bone. In this way, the frictional engagement surfaces can be varied. The diameter of the bone screw or fastener ranges from about 1.5 mm to about 5.5 mm, with about 1.9 mm being preferred. The outer diameter of the threaded portion ranges from about 3 mm to about 7 mm, with about 4 mm being preferred. The head will have an engagement feature of about 2.5 mm in diameter so as to provide sufficient space for the driver. The length of the screw or fastener ranges from about 6 mm to about 90 mm. The screw or fastener may be made from stainless steel, titanium or titanium alloys.

More specifically, the exterior thread 110 helically surrounds at least a portion of the internal lattice structure 102, thereby positioning the lattice structure 102 behind or below the outer edge of the exterior thread 110. The internal lattice structure 102 forms or covers at least a part of the cylindrical body 104. Further, the lattice structure 102 may also be present at the minor diameter or narrow portion 107 of the cylindrical body 104, and can be shaped as a geometric design comprising shapes such as crosses, squares, and/or triangles in every direction, or shaped in more of an organic design where the indices are random and protrude in all directions, at different angles and thicknesses. The lattices can be provided in repeating patterns, random patterns or may have spaces between areas of the areas covered by the lattice.

Furthermore, FIG. 2 discloses the internal lattice structure 102 which is typically positioned near the distal tip 116 at the second end 108 of the bone screw device or medical fastener 100. However, any portion of the cylindrical body 104, or the entire cylindrical body 104 can be shaped as an internal lattice structure 102, depending on the preferences or requirements of a user. More specifically, the improved bone screw device or medical fastener 100 typically positions the internal lattice structure 102 in the distal tip 116, so that bone is channeled into the internal bone lattice structure 102 (e.g., by cutting flutes 109), thereby helping to secure the distal tip 116 of the bone screw device 100 into the patient's pedicle, bone or joint during surgery.

FIG. 3 shows the medical fastener or bone screw 300 with thread zones 301 and 307 provided on the body 306 of the fastener 300. FIG. 3 provides first and second zones 302 and 304 of lattice elements or works disposed on different areas of the exterior surface of the body 306. Each of the first and second zones of lattice elements or works 302 and 304 contain different patterns of lattice elements 303 and 305 which may be formed from any shape or configurations. By providing lattice work in different areas, the bone screw or fastener can be tailored specifically to meet the requirements of the particular procedure to be performed by the medical professional.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A medical fastener comprising:
a body having a first end, a second end and an exterior surface;
the first end having an engagement feature for engaging with an insertion tool, and the second end having a tip;
a threaded portion provided on at least a portion of the body, wherein the threaded portion extends around the exterior surface; and
a lattice element provided on at least a portion of the exterior surface of the body.

2. The medical fastener as recited in claim 1, wherein the lattice element is provided near the tip.

3. The medical fastener as recited in claim 2, wherein the lattice element is provided between a pair of threads of the threaded portion.

4. The medical fastener as recited in claim 1, wherein the threaded portion extends from the tip to a head.

5. The medical fastener as recited in claim 1, wherein the lattice element is formed from a plurality of different shapes and configurations.

6. The medical fastener as recited in claim 1, wherein the lattice element extends throughout the threaded portion.

7. The medical fastener as recited in claim 1, wherein the lattice element is provided substantially along an entire length of the medical fastener.

8. The medical fastener as recited in claim 1, wherein the lattice element and the threaded portion are formed by an additive manufacturing process.

9. The medical fastener as recited in claim 1, wherein the medical fastener is formed from one of a stainless steel, a titanium or a titanium alloy.

10. The medical fastener as recited in claim 9, wherein the body has a width that ranges from about 1.5 mm to about 5.5 mm, the threaded portion has a diameter that ranges from about 3 mm to about 7 ram, and the medical fastener has a length that ranges from about 6 mm to about 90 mm.

11. A bone screw comprising:
a body having an exterior surface and a lattice work provided over a portion of the exterior surface of the body; and
a tip disposed at a first end of the body, wherein the tip is comprised of a lattice work area for funneling a bone material, and further wherein the lattice work area of the tip and the lattice work on the portion of the exterior surface of the body promote bone growth and integration with the bone screw.

12. The bone screw as recited in claim 11, wherein the lattice work is created on the bone screw by an additive manufacturing process.

13. The bone screw as recited in claim 11, wherein the exterior surface of the body further comprises a threaded portion covering at least a portion of the exterior surface of the body.

14. The bone screw as recited in claim 13, wherein the lattice work provided on the portion of the exterior surface of the body is provided in an area of the threaded portion.

15. The bone screw as recited in claim 14, wherein the threaded portion has a height which is greater than a height of the lattice work.

16. The bone screw as recited in claim 11, wherein the lattice work is formed from different shapes or configurations.

17. The bone screw as recited in claim 11, wherein the body has a width that ranges from about 1.5 mm to about 5.5 mm, the threaded portion has a diameter that ranges from about 3 mm to about 7 mm and the medical fastener has a length which ranges from about 6 mm to about 90 mm.

18. The bone screw as recited in claim 11, wherein the bone screw is formed from one of stainless steel, titanium or titanium alloys.

19. The bone screw as recited in claim 11, wherein the lattice works are formed in first and second zones on the exterior surface of the body.

20. A bone screw comprising:
a body having a generally cylindrical shape and having an exterior surface, the body having a head and a tip at opposite ends to one another;
the exterior portion of the body having a series of threads extending generally helically around the exterior surface of the body;
a lattice work composed of a plurality of lattice element; and
the lattice work disposed near the tip and between each thread of the series of threads and the lattice work having a height less than a height of each of the threads of the series of threads.
